(19) United States
(12) Patent Application Publication
(10) Pub. No.: US 2022/0244213 A1
(43) Pub. Date: Aug. 4, 2022

(54) SENSOR ELEMENT AND GAS SENSOR

(71) Applicant: NGK Spark Plug Co., LTD., Nagoya-shi (JP)

(72) Inventors: Yoshihiro NAKANO, Nagoya-shi (JP); Tetsuo YAMADA, Nagoya-shi (JP)

(73) Assignee: NGK Spark Plug Co., LTD., Nagoya-shi (JP)

(21) Appl. No.: 17/623,746

(22) PCT Filed: Sep. 28, 2020
(86) PCT No.: PCT/JP2020/036565

§ 371 Date: Dec. 29, 2021

(30) Foreign Application Priority Data
Feb. 17, 2020 (JP) 2020-023992

(51) Int. Cl. G01N 27/30; G01N 33/00; G01N 27/333; G01M 15/10

(52) U.S. Cl. CPC G01N 27/301; G01M 15/102; G01N 27/333; G01N 33/0037

(57) ABSTRACT

A sensor element comprising a first electrode containing Au as a main component, a second electrode and a solid electrolyte body. The first electrode and the second electrode are disposed to face each other while sandwiching the solid electrolyte body therebetween. The first electrode has a first electrode portion, which faces the second electrode while sandwiching the solid electrolyte body in cooperation with the second electrode, and a first lead portion. The solid electrolyte body has an uneven portion in a surface region facing the first electrode portion, the uneven portion having a plurality of protrusions. The first electrode portion is in direct contact with the uneven portion. An insulating layer whose surface has an uneven shape is formed on a surface of the solid electrolyte body facing the first lead portion. The first lead portion is in direct contact with the insulating layer.

### CROSS-REFERENCE TO RELATED PATENT APPLICATIONS

This application is a U.S. National Phase application under 35 U.S.C. § 371 of International Patent Application No. PCT/JP2020/036565 filed on Sep. 28, 2020 and claims the benefit of priority to Japanese Patent Application No. 2020-023992 filed on Feb. 17, 2020, the contents of both of which are incorporated herein by reference in their entireties. The International Application was published in Japanese on Aug. 26, 2021 as International Publication No. WO/2021/166312 under PCT Article 21(2).

### FIELD OF THE INVENTION

The present invention relates to a sensor element and a gas sensor suitably used for measurement of the concentration of, for example, ammonia.

### BACKGROUND OF THE INVENTION

In recent years, a urea SCR (Selective Catalytic Reduction) system has attracted attention as a technique for removing nitrogen oxides (NOx) contained in exhaust gas discharged from an internal combustion engine such as a diesel engine. The urea SCR system removes nitrogen oxides contained in exhaust gas by causing ammonia (NH₃) and nitrogen oxides (NOx) to chemically react with each other, thereby reducing the nitrogen oxides to nitrogen (N₂).

In this urea SCR system, when the amount of ammonia supplied for nitrogen oxides becomes excessive, exhaust gas containing ammonia not having reacted may be discharged to the outside. In order to prevent discharge of such ammonia, the urea SCR system has employed a multi-gas sensor which can measure the concentrations of a plurality of types of gases and which includes a sensor element for measuring the concentration of ammonia contained in exhaust gas (see, for example, Japanese Patent Application Laid-Open (kokai) No. 2019-174146).

Incidentally, in the ammonia sensor element, a detection electrode and a reference electrode are provided so as to face each other while sandwiching a solid electrolyte body therebetween. The detection electrode contains Au as a main component, and ammonia gas does not combust easily on its surface. Ammonia gas combusts on the reference electrode. The ammonia sensor element detects the concentration of ammonia on the basis of a change in electromotive force between the two electrodes in accordance with a mixed potential scheme.

### PRIOR ART DOCUMENT

### Patent Document

Patent Document 1: Japanese Patent Application Laid-Open (kokai) No. 2019-174146

### Problem to be Solved by the Invention

However, there is a problem that the electrode containing Au is high in surface tension, and therefore, the degree of adhesion between the electrode and the solid electrolyte body is low. Since this problem occurs not only at an electrode portion functioning as an electrode but also at a lead portion connected to the electrode portion, enhancement of adhesion between the lead portion and the solid electrolyte body has been demanded.

Moreover, a mixed-potential-type sensor has a problem that, when the lead portion is in contact with the solid electrolyte body, an electrode reaction occurs, and the voltage of the electrode portion drops accordingly, which leads to deterioration of measurement accuracy.

The present invention has been accomplished to solve the above-described problem, and an object of the present invention is to provide a sensor element and a gas sensor in which a lead portion of an electrode containing Au as a main component is adhered to a solid electrolyte body in an improved manner and deterioration of detection accuracy is prevented.

### SUMMARY OF THE INVENTION

### Means for Solving the Problem

In order to solve the above problem, a sensor element of the present invention comprises; a first electrode containing Au as a main component, a second electrode, and a solid electrolyte body including an insulating layer with a surface that has an uneven shape, the first electrode and the second electrode being disposed to face each other at least partially while sandwiching the solid electrolyte body therebetween. The first electrode has a first electrode portion and a first lead portion. The first electrode portion faces the second electrode while sandwiching the solid electrolyte body in cooperation with the second electrode. The first lead portion is electrically connected to the first electrode portion and extends from the first electrode portion. The solid electrolyte body has an uneven portion in a surface region facing the first electrode portion. The uneven portion has a plurality of protrusions. The first electrode portion is in direct contact with the uneven portion. The insulating layer is formed on a surface of the solid electrolyte body facing the first lead portion, and the first lead portion is in direct contact with the insulating layer.

The first electrode portion containing Au is high in surface tension, and the degree of adhesion between the first electrode portion and the solid electrolyte body is low. In order to overcome such a drawback, an uneven portion is provided on the solid electrolyte body. As a result, the first electrode portion tightly adheres to the uneven portion due to the anchor effect, whereby the adhesion between the first electrode portion and the solid electrolyte body is enhanced. Also, the first lead portion containing Au is similarly high in surface tension, and the degree of adhesion between the first lead portion and its counterpart member is low.

In order to overcome such a drawback, an uneven surface is provided on the insulating layer. As a result, the first lead portion tightly adheres to the surface due to the anchor effect, whereby the adhesion between the first lead portion and the insulating layer, which is the counterpart member, is enhanced.

Further, since the insulating layer is present between the first lead portion and the solid electrolyte body, the first lead portion does not come into contact with the solid electrolyte body in the sensor element, which is a mixed-potential-type sensor (detection cell). Therefore, it is possible to prevent occurrence of an electrode reaction, via the solid electrolyte body, between the first lead portion and the reference electrode portion facing the first lead portion, thereby preventing deterioration of detection accuracy.

In the sensor element of the present invention, the uneven shape of the insulating layer may correspond to the contour of the uneven portion.

In this sensor element, since the uneven shape of the insulating layer follows the contour of the uneven portion, the surface of the insulating layer has a shape which resembles (reflects) the shape of the uneven portion and which is approximately the same as that of the uneven portion, the adhesion between the solid electrolyte body and the insulating layer is also enhanced as compared with the case where the insulating layer is formed on an even (smooth) surface of the solid electrolyte body.

Meanwhile, if the uneven shape of the insulating layer does not correspond to the contour of the uneven portion and the insulating layer is thick enough to fill the difference in height of the uneven portion, the surface of the insulating layer becomes flat, and the degree of adhesion between the insulating layer and the first lead portion decreases. Also, the thickness of the insulating layer is excessively large, and a step between the uneven portion, which serves as a groundwork for the first electrode portion, and the surface of the insulating layer, which serves as a groundwork for the first lead portion, becomes large. As a result, a step of a connection portion between the first electrode portion and the first lead portion becomes large, and therefore, wire breakage may occur between the first electrode portion and the first lead portion.

In the sensor element of the present invention, the uneven portion may be also formed in a surface region of the solid electrolyte body facing the first lead portion, and the insulating layer may have a thickness smaller than a difference in height of the protrusions.

In this sensor element, the surface of the insulating layer has an uneven shape which reflects the shape of the uneven portion without fail. Therefore, the adhesion between the first lead portion and the insulating layer is enhanced without fail, and the adhesion between the solid electrolyte body and the insulating layer is enhanced without fail.

In the sensor element of the present invention, the first electrode may contain a component of the solid electrolyte body.

In this sensor element, the adhesion between the first electrode and the solid electrolyte body is enhanced.

In the sensor element of the present invention, the protrusions may be spherical protrusions, and the spherical protrusions may have a mean particle diameter of 10 to 50 μm.

In this sensor element, the spherical protrusions form an uneven shape having an appropriate difference in height. Therefore, the anchor effect can be obtained without fail. Also, since the difference in height of the uneven shape is not excessively large, the step of the connection portion between the first electrode portion and the first lead portion becomes small, and it is possible to prevent occurrence of wire breakage between the first electrode portion and the first lead portion.

In the sensor element of the present invention, the second electrode may contain Pt as a main component, the first electrode may serve as a detection electrode, and the second electrode may serve as a reference electrode; and the first electrode, the second electrode, and the solid electrolyte body may constitute a mixed-potential-type ammonia detection cell.

This sensor element can realize an element for detecting ammonia.

The sensor element of the present invention may constitute a multi-gas sensor element comprising the sensor element and an NOx sensor section for measuring the concentration of nitrogen oxides in a gas under measurement.

This sensor element can realize an element for detecting ammonia and NOx.

The gas sensor of the present invention comprises the sensor element, and a metallic shell which holds the sensor element.

### Effect of the Invention

According to the present invention, there can be obtained a sensor element and a gas sensor in which a lead portion of an electrode containing Au as a main component is adhered to a solid electrolyte body in an improved manner and deterioration of detection accuracy is prevented.

### BRIEF DESCRIPTION OF THE DRAWINGS

FIG. 1 is a sectional view of a multi-gas sensor taken along a longitudinal direction thereof.

FIG. 2 is a block diagram showing the configurations of the multi-gas sensor and a gas sensor control apparatus.

FIG. 3 is a sectional view showing the structures of a first ammonia sensor section and a second ammonia sensor section.

FIG. 4 is a plan view showing the positional relation among the first ammonia sensor section, the second ammonia sensor section, and an oxygen concentration detection cell.

FIG. 5 is a perspective view of the first ammonia sensor section.

FIG. 6 is an exploded perspective view of the first ammonia sensor section.

FIG. 7 is a plan view of the first ammonia sensor section as viewed in direction A in FIG. 5.

FIG. 8 is a sectional view of the first ammonia sensor section taken along line B-B in FIG. 5.

FIG. 9 is a schematic sectional view of a first detection electrode and its vicinity in the case where an insulating layer has a thickness smaller than a difference in height of an uneven portion.

FIG. 10 is a schematic sectional view of the first detection electrode and its vicinity in the case where the insulating layer has a thickness larger than the difference in height of the uneven portion.

FIG. 11 is a photograph showing an actual sectional SEM image taken along a direction in which a first lead portion extends.

FIG. 12 is a photograph showing an actual SEM image of the surface of the uneven portion.

### DETAILED DESCRIPTION OF THE INVENTION

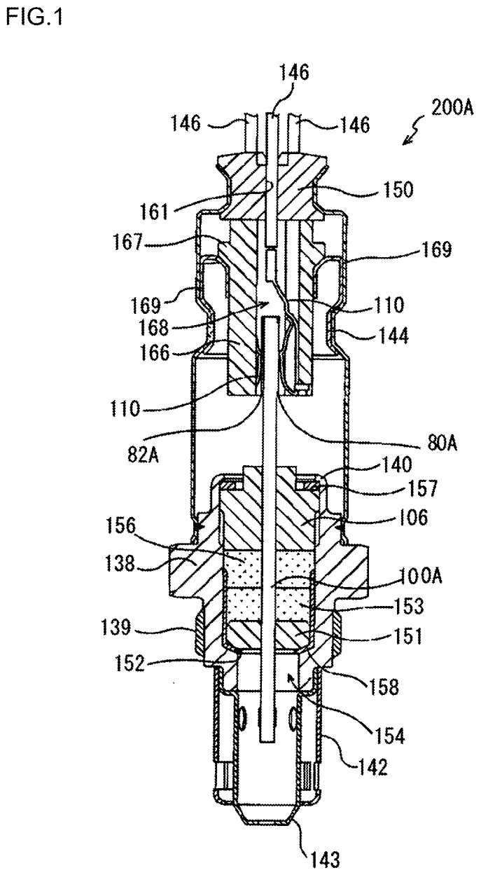

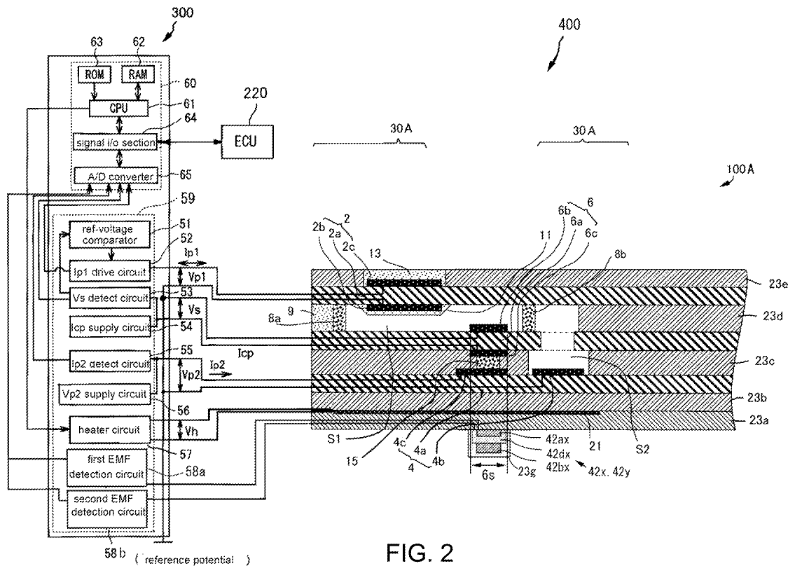

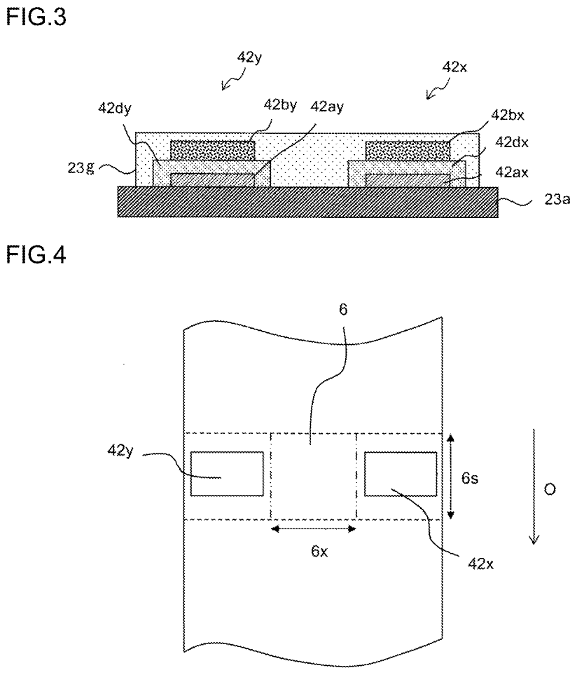

An embodiment of the present invention will be described with reference to FIGS. 1 to 4. FIG. 1 is a sectional view of a multi-gas sensor 200A taken along a longitudinal direction thereof. FIG. 2 is a block diagram showing the configuration of a multi-gas sensor apparatus 400. FIG. 3 is a sectional view showing the structures of a first ammonia sensor section 42x and a second ammonia sensor section 42y. FIG. 4 is a plan view showing the positional relation among the first ammonia sensor section 42x, the second ammonia sensor section 42y, and an oxygen concentration detection cell 6.

The multi-gas sensor 200A corresponds to the “gas sensor” in the claims, and the first ammonia sensor section 42x and the second ammonia sensor section 42y correspond to the “sensor element” in the claims. Also, a multi-gas sensor element section 100A, which will be described later, corresponds to the “multi-gas sensor element” in the claims.

The multi-gas sensor apparatus 400 is an assembly formed by connecting the multi-gas sensor 200A to a control apparatus (controller) 300, which will be described later.

The multi-gas sensor apparatus 400 of the present embodiment is used in a urea SCR system for removing nitrogen oxides (NOx) contained in exhaust gas (gas under measurement) discharged from a diesel engine. More specifically, the multi-gas sensor apparatus 400 measures the concentrations of nitrogen monoxide (NO), nitrogen dioxide (NO₂), and ammonia which are contained in exhaust gas after NOx contained in the exhaust gas is reacted with ammonia (urea).

The engine to which the multi-gas sensor apparatus 400 of the present embodiment is applied may be the above-mentioned diesel engine or a gasoline engine. No particular limitation is imposed on the type of an engine to which the multi-gas sensor apparatus 400 of the present embodiment is applied.

As shown in FIG. 1, the multi-gas sensor 200A is an assembly including the multi-gas sensor element section 100A for detecting the concentration of ammonia and the concentration of NOx. The multi-gas sensor 200A includes the multi-gas sensor element section 100A, a metallic shell 138, a ceramic sleeve 106, an insulating contact member 166, and a plurality of connection terminals 110 (only two of them are shown in FIG. 1). The multi-gas sensor element section 100A has a plate-like shape and extends in the axial direction. The metallic shell 138 has a tubular shape and has a screw portion 139 formed on an outer surface thereof and used for fixation to an exhaust pipe. The ceramic sleeve 106 has a tubular shape and is disposed to surround the circumference of the multi-gas sensor element section 100A. The insulating contact member 166 has a contact insertion hole 168 extending therethrough in the axial direction and is disposed in such a manner that the inner wall surface of the contact insertion hole 168 surrounds the circumference of a rear end portion of the multi-gas sensor element section 100A. The connection terminals 110 are disposed between the multi-gas sensor element section 100A and the insulating contact member 166.

The metallic shell 138 has a generally tubular shape, has a through hole 154 extending therethrough in the axial direction, and has a ledge portion 152 projecting inward in the radial direction of the through hole 154. The metallic shell 138 holds the multi-gas sensor element section 100A in the through hole 154 in such a manner that the forward end of the multi-gas sensor element section 100A is located on the forward end side and externally of the through hole 154, and electrode terminal portions 80A and 82A of the multi-gas sensor element section 100A are located on the rear end side and externally of the through hole 154. The ledge portion 152 is formed to have an inward taper surface which inclines in relation to a plane perpendicular to the axial direction.

An annular ceramic holder 151, powder charged layers 153 and 156 (hereinafter also referred to as talc rings 153 and 156), and the above-described ceramic sleeve 106 are stacked in the through hole 154 of the metallic shell 138 in this order from the forward end side toward the rear end side, so that these members surround the circumference of the multi-gas sensor element section 100A. Also, a crimp packing 157 is disposed between the ceramic sleeve 106 and a rear end portion 140 of the metallic shell 138, and a metallic holder 158 is disposed between the ceramic holder 151 and the ledge portion 152 of the metallic shell 138. The metallic holder 158 holds the talc ring 153 and the ceramic holder 151 and maintains gastightness. Notably, the rear end portion 140 of the metallic shell 138 is crimpled so as to press the ceramic sleeve 106 toward the forward end side via the crimp packing 157.

Meanwhile, a double protector is attached to the outer circumference of a forward-end-side (lower-side in FIG. 1) portion of the metallic shell 138 by means of, for example, welding so as to cover the protruding portion of the multi-gas sensor element section 100A. The double protector is composed of an outer protector 142 and an inner protector 143 which are made of a metal (e.g., stainless steel) and have a plurality of holes.

An outer casing 144 is fixed to the outer circumference of a rear end portion of the metallic shell 138. Also, a grommet 150 is disposed in a rear-end-side (upper-side in FIG. 1) opening of the outer casing 144. The grommet 150 has lead wire insertion holes 161 into which a plurality of lead wires 146 (only three lead wires are shown in FIG. 1) are inserted. The lead wires 146 are electrically connected to the electrode terminal portions 80A and 82A of the multi-gas sensor element section 100A. Notably, for simplification, in FIG. 1, the electrode terminal portions on the front and back surfaces of the multi-gas sensor element section 100A are collectively denoted by reference numerals 80A and 82A, respectively. However, in actuality, a plurality of electrode terminal portions are formed on each of the front and back surfaces of the multi-gas sensor element section 100A, and the number of the electrode terminal portions corresponds to the number of electrodes of an NOx sensor section 30A, which will be described later, and the first and second ammonia sensor sections 42x and 42y, etc.

The insulating contact member 166 is disposed on the rear-end side (the upper side in FIG. 1) of the multi-gas sensor element section 100A protruding from the rear end portion 140 of the metallic shell 138. Notably, the insulating contact member 166 is disposed around the electrode terminal portions 80A and 82A formed on the front and back surfaces of a rear end portion of the multi-gas sensor element section 100A. The insulating contact member 166 is formed into a tubular shape with the contact insertion hole 168 extending therethrough in the axial direction and has a flange portion 167 protruding radially outward from the outer surface of the insulating contact member 166. The flange portion 167 abuts against the outer casing 144 through a holding member 169, whereby the insulating contact member 166 is held inside the outer casing 144. The connection terminals 110 on the insulating contact member 166 side are electrically connected to the electrode terminal portions 80A and 82A of the multi-gas sensor element section 100A, so that the electrode terminal portions 80A and 82A electrically communicate with the outside through the lead wires 146.

FIG. 2 is a block diagram showing the configuration of the multi-gas sensor apparatus 400 according to the embodiment of the present invention. Notably, in FIG. 2, for convenience of description, only the longitudinal section of the multi-gas sensor element section 100A contained in the multi-gas sensor 200A is shown.

The multi-gas sensor apparatus 400 includes the control apparatus (controller) 300 and the multi-gas sensor 200A (multi-gas sensor element section 100A) connected thereto. The control apparatus 300 is mounted on an unillustrated vehicle including an internal combustion engine, and the control apparatus 300 is electrically connected to an ECU 220. Notably, ends of the lead wires 146 extending from the multi-gas sensor 200A are connected to a connector, and the connector is electrically connected to a connector provided on the control apparatus 300.

Next, the structure of the multi-gas sensor element section 100A will be described. The multi-gas sensor element section 100A includes the NOx sensor section 30A having the same structure as a known NOx sensor, and two ammonia sensor sections; i.e., the first ammonia sensor section 42x and the second ammonia sensor section 42y. As will be described in detail later, the first ammonia sensor section 42x and the second ammonia sensor section 42y are formed on an outer surface of the NOx sensor section 30A.

The NOx sensor section 30A has a structure including an insulating layer 23e, a first solid electrolyte layer 2a, an insulating layer 23d, a third solid electrolyte layer 6a, an insulating layer 23c, a second solid electrolyte layer 4a, and insulating layers 23b and 23a, which are stacked in this order. A first measurement chamber S1 is defined between the first solid electrolyte layer 2a and the third solid electrolyte layer 6a. Exhaust gas is externally introduced into the first measurement chamber S1 through a first diffusion resistor element 8a disposed at the left end (inlet) of the first measurement chamber S1. Notably, a protection layer 9 formed of a porous material is disposed on the outer side of the first diffusion resistor element 8a.

A second diffusion resistor element 8b is disposed at an end of the first measurement chamber S1 opposite the inlet. A second measurement chamber S2 (which corresponds to the “NOx measurement chamber” of the present invention) communicating with the first measurement chamber S1 is defined on the right side of the first measurement chamber S1, with the second diffusion resistor element 8b intervening between the first measurement chamber S1 and the second measurement chamber S2. The second measurement chamber S2 is formed between the first solid electrolyte layer 2a and the second solid electrolyte layer 4a and penetrates the third solid electrolyte layer 6a.

An elongated plate-shaped heating resistor element 21 extending along the longitudinal direction of the multi-gas sensor element section 100A is embedded between the insulating layers 23b and 23a. The heating resistor element 21 has a heat generating portion provided on the forward end side in the axial direction (the longitudinal direction), and a pair of lead portions extending from the heat generating portion toward the rear end side in the axial direction. The heating resistor element 21 and the insulating layers 23b and 23a constitute a heater. This heater is used to heat the gas sensor to an activation temperature, thereby increasing the oxygen ion conductivity of each solid electrolyte layer for stable operation.

The insulating layers 23a, 23b, 23c, 23d, and 23e are formed mainly of alumina, and the first diffusion resistor element 8a and the second diffusion resistor element 8b are formed of a porous material such as alumina. The heating resistor element 21 is formed of, for example, platinum. The heat generating portion of the heating resistor element 21 is formed into a meandering pattern. However, the pattern of the heat generating portion is not limited thereto.

The first pumping cell 2 includes a first solid electrolyte layer 2a formed mainly of zirconia having oxygen ion conductivity, and an inner first pumping electrode 2b and an outer (counter) first pumping electrode 2c disposed to sandwich the first solid electrolyte layer 2a. The inner first pumping electrode 2b faces the first measurement chamber S1. Each of the inner first pumping electrode 2b and the outer first pumping electrode 2c is formed mainly of platinum, and the surface of the inner first pumping electrode 2b is covered with a protection layer 11 formed of a porous material.

A portion of the insulating layer 23e corresponding to the upper surface of the outer first pumping electrode 2c is removed to form a cutout, and a porous material 13 is charged into the cutout. The porous material 13 establishes communication between the outer first pumping electrode 2c and the outside, thereby allowing a gas (oxygen) to enter and leave.

The oxygen concentration detection cell 6 includes a third solid electrolyte layer 6a formed mainly of zirconia, and a detection electrode 6b and a reference electrode 6c disposed to sandwich the third solid electrolyte layer 6a. The detection electrode 6b faces the first measurement chamber S1 on the downstream side of the inner first pumping electrode 2b. Each of the detection electrode 6b and the reference electrode 6c is formed mainly of platinum.

Notably, a cutout is formed in the insulating layer 23c in such a manner that the reference electrode 6c in contact with the third solid electrolyte layer 6a is disposed in the cutout. A porous material is charged into the cutout, whereby a reference oxygen chamber 15 is formed. When a constant weak current is supplied to the oxygen concentration detection cell 6 beforehand by using an Icp supply circuit 54, the oxygen concentration detection cell 6 feeds oxygen from the first measurement chamber S1 into the reference oxygen chamber 15. The oxygen within the reference oxygen chamber 15 serves as an oxygen reference.

The second pumping cell 4 includes a second solid electrolyte layer 4a formed mainly of zirconia, an inner second pumping electrode 4b disposed on a surface region of the second solid electrolyte layer 4a facing the second measurement chamber S2, and a second pumping counter electrode 4c serving as a counter electrode. Each of the inner second pumping electrode 4b and the second pumping counter electrode 4c is mainly formed of platinum.

Notably, the second pumping counter electrode 4c is disposed in a cutout portion of the insulating layer 23c located on the second solid electrolyte layer 4a. The second pumping counter electrode 4c is opposed to the reference electrode 6c and faces the reference oxygen chamber 15.

The inner first pumping electrode 2b, the detection electrode 6b, and the inner second pumping electrode 4b are connected to a reference potential.

Next, the first ammonia sensor section 42x and the second ammonia sensor section 42y, which are the two ammonia sensor sections, will be described.

As shown in FIG. 3, the multi-gas sensor element section 100A includes the first ammonia sensor section 42x and the second ammonia sensor section 42y, which are separated from each other in the width direction.

The first ammonia sensor section 42x and the second ammonia sensor section 42y are formed on the insulating layer 23a, which forms the outer surface (lower surface) of the NOx sensor section 30A. More specifically, in the first ammonia sensor section 42x, a first reference electrode 42ax is formed on the insulating layer 23a, and a first solid electrolyte layer 42dx is formed to cover the upper and side surfaces of the first reference electrode 42ax. Moreover, a first detection electrode 42bx is formed on the surface of the first solid electrolyte layer 42dx. The ammonia concentration in the gas under measurement is detected from a change in the electromotive force between the first reference electrode 42ax and the first detection electrode 42bx.

Similarly, in the second ammonia sensor section 42y, a second reference electrode 42ay is formed on the insulating layer 23a, and a second solid electrolyte layer 42dy is formed to cover the upper and side surfaces of the second reference electrode 42ay. Moreover, a second detection electrode 42by is formed on the surface of the second solid electrolyte layer 42dy.

The first detection electrode 42bx and the second detection electrode 42by correspond to the “first electrode” in the claims. The first reference electrode 42ax and the second reference electrode 42ay correspond to the “second electrode” in the claims.

In the present embodiment, the NOx detection section and the first and second ammonia sensor sections 42x and 42y are disposed to sandwich the heater (the heating resistor element 21, the insulating layer 23b, and the insulating layer 23a) in the stacking direction. However, the NOx detection section and the two ammonia sensor sections 42x and 42y may be disposed on one side of the heater in the stacking direction.

In the present embodiment, the first ammonia sensor section 42x includes the solid electrolyte body 42dx and the pair of electrodes 42ax and 42bx provided on opposite surfaces of the solid electrolyte body 42dx, and the second ammonia sensor section 42y includes the solid electrolyte body 42dy and the pair of electrodes 42ay and 42by provided on opposite surfaces of the solid electrolyte body 42dy. Of the two pairs of electrodes, the first reference electrode 42ax and the second reference electrode 42ay are disposed on the outer surface of the NOx sensor section 30A.

The first ammonia sensor section 42x and the second ammonia sensor section 42y are integrally covered with a protection layer 23g formed of a porous material.

The protection layer 23g prevents adhesion of poisoning substances to the first detection electrode 42bx and the second detection electrode 42by and adjusts the diffusion rate of the gas under measurement flowing into the first ammonia sensor section 42x and the second ammonia sensor section 42y from the outside. The protection layer 23g is formed through use of at least one type of material selected from a group consisting of alumina (aluminum oxide), spinel (MgAl₂O₄), silica alumina, and mullite. The rate of diffusion of the gas under measurement through the protection layer 23g is adjusted by adjusting the thickness, particle size, particle size distribution, porosity, material blending ratio, etc. of the protection layer 23g.

Notably, the present invention is not limited to the case where the protection layer 23g is provided. The protection layer 23g may be omitted to expose the first ammonia sensor section 42x, the second ammonia sensor section 42y, etc. Also, in the case where the ratio of sensitivity between the first ammonia sensor section 42x and the second ammonia sensor section 42y is adjusted by the protection layer 23g, unlike the above-described embodiment, separate protection layers may be individually provided for the first ammonia sensor section 42x and the second ammonia sensor section 42y.

Each of the first detection electrode 42bx and the second detection electrode 42by is an electrode of a type in which ammonia gas does not combust easily on the electrode surface. Ammonia passes through the detection electrode 42bx (42by) and reacts (electrode reaction) with oxygen ions at the interface between the detection electrode 42bx (42by) and the reference electrode 42ax (42ay) located thereunder, whereby the concentration of ammonia is detected.

The first detection electrode 42bx and the second detection electrode 42by may be formed from a material containing Au as a main component (for example, 70 mass % or more). The first reference electrode 42ax and the second reference electrode 42ay may be formed from a material containing Pt only or containing Pt as a main component (for example, 70 mass % or more).

However, no particular limitation is imposed on the first reference electrode 42ax and the second reference electrode 42ay, so long as the first reference electrode 42ax and the second reference electrode 42ay have compositions determined such that combustion of ammonia gas on the electrode surface occurs more easily as compared with the first detection electrode 42bx and the second detection electrode 42by.

When the first detection electrode 42bx and the second detection electrode 42by contain the component of the first solid electrolyte body 42dx and the second detection electrode 42by in addition to Au, the adhesion to the first solid electrolyte body 42dx and the second detection electrode 42by is enhanced.

Each of the first solid electrolyte body 42dx and the second solid electrolyte body 42dy is formed of, for example, partially stabilized zirconia (YSZ). Also, the first reference electrode 42ax and the second reference electrode 42ay may have the same composition as the reference electrode 42a, and the first detection electrode 42bx and the second detection electrode 42by may have the same composition as the detection electrode 42b.

In the present embodiment, the impedance of the oxygen concentration detection cell 6 is measured, and the heater (the heating resistor element 21) is activated for heating on the basis of the measured impedance. Therefore, the temperature of the multi-gas sensor element section 100A is maintained at the most stable value (temperature estimable value) in the vicinity of the oxygen concentration detection cell 6.

Therefore, as shown in FIG. 4, the first ammonia sensor section 42x and the second ammonia sensor section 42y are disposed in such a manner that at least a portion of each of the first ammonia sensor section 42x and the second ammonia sensor section 42y overlaps with a region 6s between the opposite ends of the oxygen concentration detection cell 6 in the direction of the axial line O. As a result, the two ammonia sensor sections 42x and 42y are maintained at a more stable temperature.

The oxygen concentration detection cell 6 is disposed in a central portion of the multi-gas sensor element section 100A in the width direction (direction perpendicular to the axial direction), and the first ammonia sensor section 42x and the second ammonia sensor section 42y are disposed on opposite sides (in the width direction) of a width-direction region 6X in which the oxygen concentration detection cell 6 is formed.

Next, referring back to FIG. 2, an example of the configuration of the control apparatus 300 will be described. The control apparatus 300 includes a (analog) control circuit 59 and a microcomputer 60 disposed on a circuit board. The microcomputer 60, which controls the entirety of the control apparatus 300, includes a CPU (central processing unit) 61, a RAM 62, a ROM 63, a signal input/output section 64, an A/D converter 65, and an unillustrated clock. Programs stored in the ROM, etc. in advance are executed by the CPU.

The control circuit 59 includes a reference voltage comparison circuit 51, an Ip1 drive circuit 52, a Vs detection circuit 53, an Icp supply circuit 54, an Ip2 detection circuit 55, a Vp2 application circuit 56, and a heater circuit 57, as well as a first electromotive force detection circuit 58a and a second electromotive force detection circuit 58b which detect electromotive forces of the first ammonia sensor section 42x and the second ammonia sensor section 42y, respectively. These circuits will be described in detail later.

The control circuit 59 controls the NOx sensor section 30A, detects the first and second pumping currents Ip1 and Ip2 flowing through the NOx sensor section 30A, and outputs the detected first and second pumping currents Ip1 and Ip2 to the microcomputer 60.

The first electromotive force detection circuit 58a detects the ammonia concentration output (electromotive force) between the electrodes of the first ammonia sensor section 42x, and outputs the detected ammonia concentration output to the microcomputer 60. Similarly, the second electromotive force detection circuit 58b detects the ammonia concentration output (electromotive force) between the electrodes of the second ammonia sensor section 42y, and outputs the detected ammonia concentration output to the microcomputer 60.

Specifically, the outer first pumping electrode 2c of the NOx sensor section 30A is connected to the Ip1 drive circuit 52, and the reference electrode 6c is connected to the Vs detection circuit 53 and the Icp supply circuit 54 in parallel. The second pumping counter electrode 4c is connected to the Ip2 detection circuit 55 and the Vp2 application circuit 56 in parallel. The heater circuit 57 is connected to the heater (specifically, the heating resistor element 21).

The pair of electrodes 42ax and 42bx of the first ammonia sensor section 42x are connected to the first electromotive force detection circuit 58a. Similarly, the pair of electrodes 42ay and 42by of the second ammonia sensor section 42y are connected to the second electromotive force detection circuit 58b.

The circuits 51 to 57 have the following functions.

The Ip1 drive circuit 52 supplies the first pumping current Ip1 flowing between the inner first pumping electrode 2b and the outer first pumping electrode 2c and detects the first pumping current Ip1 at that time.

The Vs detection circuit 53 detects the voltage Vs between the detection electrode 6b and the reference electrode 6c, and outputs the detected voltage to the reference voltage comparison circuit 51.

The reference voltage comparison circuit 51 compares a reference voltage (for example, 425 mV) and the output (the voltage Vs) of the Vs detection circuit 53, and outputs the comparison result to the Ip1 drive circuit 52. The Ip1 drive circuit 52 controls the flow direction and magnitude of the current Ip1 such that the voltage Vs becomes equal to the above-described reference voltage, thereby adjusting the oxygen concentration within the first measurement chamber S1 to a predetermined concentration at which NOx does not decompose.

The Icp supply circuit 54 supplies a weak current Icp which flows between the detection electrode 6b and the reference electrode 6c so as to feed oxygen from the first measurement chamber S1 into the reference oxygen chamber 15, thereby exposing the reference electrode 6c to oxygen of a predetermined concentration serving as a reference.

The Vp2 application circuit 56 applies a constant voltage Vp2 (for example, 450 mV) between the inner second pumping electrode 4b and the second pumping counter electrode 4c. The constant voltage Vp2 is high enough to decompose the NOx gas contained in the gas under measurement to oxygen and N₂ gas. As a result, NOx is decomposed to nitrogen and oxygen.

The Ip2 detection circuit 55 detects the second pumping current Ip2 flowing through the second pumping cell 4 when oxygen produced as a result of decomposition of NOx is pumped out from the second measurement chamber S2 toward the second pumping counter electrode 4c through the second solid electrolyte layer 4a.

The Ip1 drive circuit 52 outputs the value of the detected first pumping current Ip1 to the A/D converter 65. The Ip2 detection circuit 55 outputs the value of the detected second pumping current Ip2 to the A/D converter 65.

The A/D converter 65 converts these values to digital values and outputs the digital values to the CPU 61 via the signal input/output section 64.

Next, an example of control performed through use of the control circuit 59 will be described. When an engine is started and electric power is supplied from an external power source, the heater operates through the heater circuit 57, whereby the first pumping cell 2, the oxygen concentration detection cell 6, and the second pumping cell 4 are heated to their activation temperatures. The Icp supply circuit 54 supplies the weak current Icp which flows between the detection electrode 6b and the reference electrode 6c so as to feed oxygen from the first measurement chamber S1 into the reference oxygen chamber 15, so that the oxygen within the reference oxygen chamber 15 serves as a reference.

When the NOx sensor section 30A is heated to an appropriate temperature by the heater, the first ammonia sensor section 42x and the second ammonia sensor section 42y on the NOx sensor section 30A are also heated to a desired temperature.

After the cells have been heated to their activation temperatures, the first pumping cell 2 pumps out oxygen contained in the gas under measurement (exhaust gas) flowing into the first measurement chamber S1. The pumped out oxygen flows from the inner first pumping electrode 2b toward the outer first pumping electrode 2c.

At that time, the oxygen concentration within the first measurement chamber S1 corresponds to the voltage (inter-terminal voltage) Vs between the electrodes of the oxygen concentration detection cell 6. Therefore, the Ip1 drive circuit 52 controls the first pumping current Ip1 flowing through the first pumping cell 2 such that the inter-terminal voltage Vs becomes equal to the above-described reference voltage. In this manner, the oxygen concentration within the first measurement chamber S1 is adjusted to a concentration at which NOx does not decompose.

The gas under measurement whose oxygen concentration has been adjusted flows further toward the second measurement chamber S2. The Vp2 application circuit 56 applies the constant voltage Vp2 between the electrodes of the second pumping cell 4 as an inter-electrode voltage (inter-terminal voltage). The constant voltage Vp2 is high enough to decompose the NOx gas contained in the gas under measurement to oxygen and N₂ gas (a voltage higher than the value of the control voltage of the oxygen concentration detection cell 6, for example, 450 mV). As a result, NOx is decomposed to nitrogen and oxygen. The second pumping current Ip2 flows through the second pumping cell 4 so that oxygen produced as a result of decomposition of NOx is pumped out from the second measurement chamber S2. At that time, since a linear relation is present between the second pumping current Ip2 and the NOx concentration, the NOx concentration in the gas under measurement can be detected on the basis of the second pumping current Ip2 detected by the Ip2 detection circuit 55.

Also, the first electromotive force detection circuit 58a detects the ammonia concentration output (electromotive force) between the pair of electrodes 42ax and 42bx, and the second electromotive force detection circuit 58b detects the ammonia concentration output (electromotive force) between the pair of electrodes 42ay and 42by. As will be described later, the ammonia concentration in the gas under measurement can be detected on the basis of the detected ammonia concentration outputs.

Next, processes for calculating various gas concentrations performed by the microcomputer 60 of the control apparatus 300 will be described.

First, the reason for provision of two ammonia sensor sections; i.e., the first ammonia sensor section 42x and the second ammonia sensor section 42y, is as follows. Namely, since the ammonia sensor sections detect not only ammonia but also NO₂, if NO₂ gas is contained in the gas to be detected in addition to ammonia, the accuracy in detecting ammonia lowers. In view of the above, two ammonia sensor sections which differ from each other in terms of the ratio between the sensitivity to ammonia and the sensitivity to NOx are provided. For two unknown concentrations of ammonia gas and NO₂ gas, individual sensitivity-depending values are detected from the two ammonia sensor sections, and therefore, the concentrations of ammonia gas and NO₂ can be calculated.

Here, the “ratio between the sensitivity of the ammonia sensor section to ammonia and the sensitivity of the ammonia sensor section to NOx” means the ratio of the ammonia detection sensitivity to the overall sensitivity to gas components (ammonia and NOx) detected by the ammonia sensor section.

Notably, in the present embodiment, since the ammonia sensor section does not detect NO gas, the “ratio between the sensitivity of the ammonia sensor section to ammonia and the sensitivity of the ammonia sensor section to NOx” is considered to be the “ratio between the sensitivity of the ammonia sensor section to ammonia and the sensitivity of the ammonia sensor section to NO₂.” Also, in the case where the ammonia sensor section does not detect NO₂ gas, the “ratio between the sensitivity of the ammonia sensor section to ammonia and the sensitivity of the ammonia sensor section to NOx” may be considered to be the “ratio between the sensitivity of the ammonia sensor section to ammonia and the sensitivity of the ammonia sensor section to NO.”

Namely, the sensor output of each ammonia sensor section is represented by F(x, y, D), where x: ammonia concentration, y: NO₂ gas concentration, and D: O₂ concentration. When two ammonia sensor sections which differ in the above-mentioned sensitivity ratio are used, two expressions F₁(mx, ny, D) and F₂(sx, ty, D) are obtained, where m, n, s, and t are coefficients. Since F₁, F₂, and D can be obtained from the sensor outputs, two unknown quantities (x, y) can be obtained from the two expressions. Specifically, y is removed from the above-described two expressions and expressions of x (expressions (1) to (3) which will be described later) are obtained, whereby the ammonia concentration can be obtained.

Notably, the ratio between the sensitivity of the first ammonia sensor section 42x to ammonia and the sensitivity of the second ammonia sensor section 42y to ammonia may change when the temperatures of the first ammonia sensor section 42x and the second ammonia sensor section 42y differ from each other. In order to overcome such a problem, as described above, the first ammonia sensor section 42x and the second ammonia sensor section 42y are disposed in such a manner that, as viewed in the axial direction, each of the first ammonia sensor section 42x and the second ammonia sensor section 42y overlaps with at least a portion of the region 6s of the oxygen concentration detection cell 6, whereby the temperatures of the first ammonia sensor section 42x and the second ammonia sensor section 42y are maintained constant within a predetermined range. As a result, the above-described change in the sensitivity ratio with temperature can be reduced.

Next, detection of NO₂ and ammonia by the first ammonia sensor section 42x and the second ammonia sensor section 42y and calculation of the concentrations of NO₂ and ammonia will be described in detail.

Electromotive force is generated between the first reference electrode 42ax and the first detection electrode 42bx of the first ammonia sensor section 42x in accordance with the concentration of ammonia contained in the gas under measurement. The first electromotive force detection circuit 58a detects the electromotive force between the first reference electrode 42ax and the first detection electrode 42bx as a first ammonia electromotive force. Similarly, electromotive force is generated between the second reference electrode 42ay and the second detection electrode 42by of the second ammonia sensor section 42y in accordance with the ammonia concentration. The second electromotive force detection circuit 58b detects the electromotive force between the second reference electrode 42ay and the second detection electrode 42by as a second ammonia electromotive force.

Various types of data (relational expressions) described below are stored in the ROM 63 of the microcomputer 60. The CPU 61 reads out the various types of data from the ROM 63 and performs various computation processes while using the value of the first pumping current Ip1, the value of the second pumping current Ip2, the first ammonia electromotive force, and the second ammonia electromotive force.

The ROM 63 stores a “first ammonia electromotive force−first ammonia concentration output relational expression,” a “second ammonia electromotive force−second ammonia concentration output relational expression,” a “first pumping current Ip1−O₂ concentration output relational expression,” a “second pumping current Ip2−NOx concentration output relational expression,” a “first ammonia concentration output & second ammonia concentration output & O₂ concentration output−corrected ammonia concentration output relational expression” (Correction expression (1): see below), a “first ammonia concentration output & second ammonia concentration output & O₂ concentration output−corrected NO₂ concentration output relational expression” (Correction expression (2)), and an “NOx concentration output & corrected ammonia concentration output & corrected NO₂ concentration output−corrected NOx concentration output relational expression” (Correction expression (3)).

Notably, the various types of data may be set in the form of predetermined relational expressions as described above or may be set in other forms (for example, tables) so long as various gas concentrations can be calculated from the outputs of the sensor. Alternatively, they may be values (relational expressions, tables, or the like) obtained through the use of a model gas whose gas concentration is known.

The “first ammonia electromotive force−first ammonia concentration output relational expression” and the “second ammonia electromotive force−second ammonia concentration output relational expression” are expressions representing the relation between the ammonia electromotive forces outputted from the first ammonia sensor section 42x and the second ammonia sensor section 42y and the ammonia concentration outputs regarding the ammonia concentration of the gas under measurement.

The “first pumping current Ip1−O₂ concentration output relational expression” is an expression representing the relation between the first pumping current Ip1 and the O₂ concentration of the gas under measurement.

The “second pumping current Ip2−NOx concentration output relational expression” is an expression representing the relation between the second pumping current Ip2 and the NOx concentration of the gas under measurement.

The “first ammonia concentration output & second ammonia concentration output & O₂ concentration output−corrected ammonia concentration output relational expression” is an expression representing the relation between the (first and second) ammonia concentration outputs affected by the oxygen concentration and the NO₂ concentration and the corrected ammonia concentration output from which the influences of the oxygen concentration and the NO₂ concentration have been removed.

The “first ammonia concentration output & second ammonia concentration output & O₂ concentration output−corrected NO₂ concentration output relational expression” is an expression representing the relation between the NO₂ concentration output affected by the oxygen concentration and the ammonia concentration and the corrected NO₂ concentration output from which the influences of the oxygen concentration and the ammonia concentration have been removed.

The “NOx concentration output & corrected ammonia concentration output & corrected NO₂ concentration output−corrected NOx concentration output relational expression” is an expression representing the relation between the NOx concentration output affected by the ammonia concentration and the NO₂ concentration and the corrected, accurate NOx concentration output from which the influences of the ammonia concentration and the NO₂ concentration have been removed.

A description will next be given of a computation process for obtaining the NOx concentration and the ammonia concentration from the first pumping current Ip1, the second pumping current Ip2, the first ammonia electromotive force EMF, and the second ammonia electromotive force EMF. This computation process is executed by the CPU 61 of the microcomputer 60.

When the first pumping current Ip1, the second pumping current Ip2, the first ammonia electromotive force, and the second ammonia electromotive force are inputted, the CPU 61 performs a computation process for obtaining the 02 concentration output, the NOx concentration output, the first ammonia concentration output, and the second ammonia concentration output. Specifically, the CPU 61 invokes the “first ammonia electromotive force−first ammonia concentration output relational expression,” the “second ammonia electromotive force−second ammonia concentration output relational expression,” the “first pumping current Ip1−O₂ concentration output relational expression,” and the “second pumping current Ip2−NOx concentration output relational expression” from the ROM 63 and then calculates the concentration outputs using these relational expressions.

Notably, the “first ammonia electromotive force−first ammonia concentration output relational expression” and the “second ammonia electromotive force−second ammonia concentration output relational expression” are set such that, over the entire possible range of EMF outputted from the first ammonia sensor section 42x and the second ammonia sensor section 42y in their use environment, an approximately linear relation is present between each of the ammonia concentration converted outputs from the sensors and the ammonia concentration in the gas under measurement. Since these conversion expressions are used for conversion, in the correction expressions below, calculation which utilizes changes in gradient and offset is possible.

After the O₂ concentration output, the NOx concentration output, the first ammonia concentration output, and the second ammonia concentration output are obtained, the CPU performs computations using the correction expressions described below to obtain the ammonia concentration and NOx concentration of the gas under measurement.

x=F(A,B,D)=(eA−c)*(jB−h−fA+d)/(eA−c−iB+g)+fA−d Correction expression (1):

y=F′(A,B,D)=(jB−h−fA+d)/(eA−c−iB+g) Correction expression (2):

z=C−ax+by Correction expression (3):

In these correction expressions, x represents the ammonia concentration, y represents the NO₂ concentration, and z represents the NOx concentration. Also, A represents the first ammonia concentration output, B represents the second ammonia concentration output, C represents the NOx concentration output, and D represents the O₂ concentration output. F and F′ in the expressions (1) and (2) represent that x is a function of A, B, and D; and y is a function of A, B, and D. Further, a and b are correction coefficients, and c, d, e, f, g, h, i, and j are coefficients calculated using the O₂ concentration output D (i.e., coefficients determined by D).

The CPU 61 obtains the ammonia concentration and the NOx concentration of the gas under measurement by substituting the first ammonia concentration output (A), the second ammonia concentration output (B), the NOx concentration output (C), and the O₂ concentration output (D) into the above-described correction expressions (1) to (3).

Notably, the correction expressions (1) and (2) are determined on the basis of the characteristics of the first ammonia sensor section 42x and the second ammonia sensor section 42y, and the correction expression (3) is determined on the basis of the characteristics of the NOx sensor section. Notably, the correction expressions (1) to (3) are merely examples, and other correction expressions, coefficients, etc. may be appropriately changed in accordance with the characteristics of gas detection.

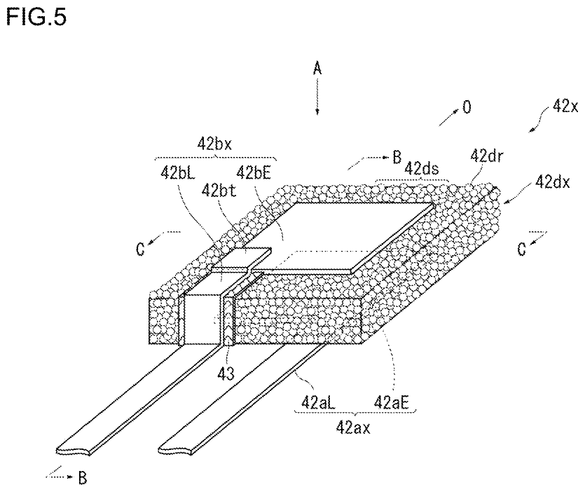

Next, the specific structure of the first ammonia sensor section 42x and the second ammonia sensor section 42y, which is the feature of the present invention, will be described with reference to FIGS. 5 to 8. Notably, the structure of the first ammonia sensor section 42x will be described below as an example. However, the second ammonia sensor section 42y has the same structure.

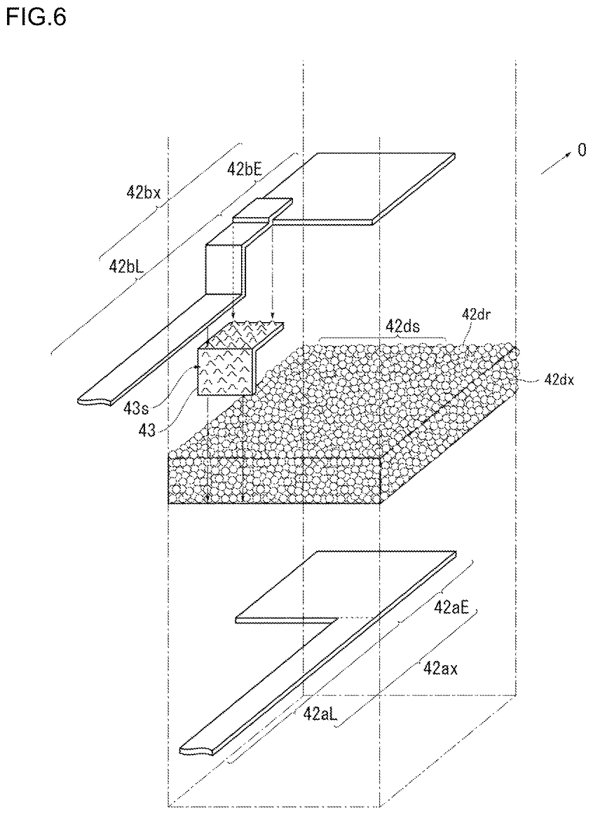

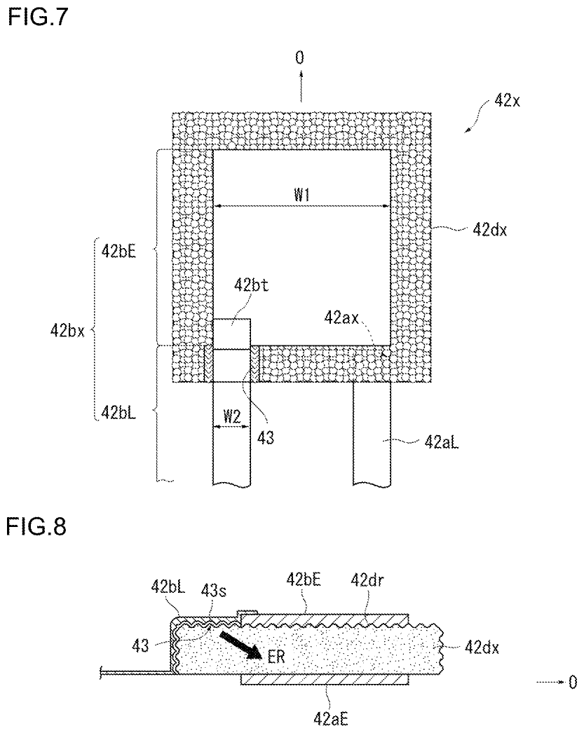

FIG. 5 is a perspective view of the first ammonia sensor section 42x. FIG. 6 is an exploded perspective view of the first ammonia sensor section 42x. FIG. 7 is a plan view of the first ammonia sensor section 42x as viewed in direction A in FIG. 5. FIG. 8 is a sectional view of the first ammonia sensor section 42x taken along line B-B in FIG. 5.

As shown in FIGS. 5 and 6, the first detection electrode 42bx and the first reference electrode 42ax are disposed in such a manner that the first detection electrode 42bx and the first reference electrode 42ax face each other at least partially while sandwiching the first solid electrolyte body 42dx therebetween.

Notably, a section taken along line C-C in FIG. 5 is shown in FIG. 3.

The first detection electrode 42bx has a first electrode portion 42bE facing the first reference electrode 42ax while sandwiching the first solid electrolyte body 42dx in cooperation with the first reference electrode 42ax, and a first lead portion 42bL electrically connected to the first electrode portion 42bE and extending in the direction of the axial line O.

Similarly, the first reference electrode 42ax has a reference electrode portion 42aE facing the first detection electrode 42bx while sandwiching the first solid electrolyte body 42dx in cooperation with the first detection electrode 42bx, and a reference lead portion 42aL electrically connected to the reference electrode portion 42aE and extending in the direction of the axial line O.

Each of the first electrode portion 42bE and the reference electrode portion 42aE functions as an electrode and has an approximately rectangular shape in the present embodiment.

The first lead portion 42bL and the reference lead portion 42aL are respectively connected to the rear ends of the first electrode portion 42bE and the reference electrode portion 42aE and extend to form a strip like shape. The first lead portion 42bL and the reference lead portion 42aL are respectively narrower than the first electrode portion 42bE and the reference electrode portion 42aE and are formed of a porous material so as to allow oxygen from the first electrode portion 42bE and the reference electrode portion 42aE to pass through the first lead portion 42bL and the reference lead portion 42aL, respectively.

Notably, as shown in FIG. 7, in order to separate the first lead portion 42bL and the reference lead portion 42aL in the width direction, in the present embodiment, the first lead portion 42bL is connected to a left rear end of the first electrode portion 42bE, and the reference lead portion 42aL is connected to a right rear end of the reference electrode portion 42aE.

As shown in FIG. 7, the boundary between the first electrode portion 42bE and the first lead portion 42bL is located at a position where the average width W2 of the first lead portion 42bL becomes equal to or less than ½ of the maximum width W1 of the first electrode portion 42bE. The average width W2 of the first lead portion 42bL is obtained by measuring, at several locations, the width of the first lead portion 42bL in the direction perpendicular to the direction (the direction of the axial line O in FIG. 7) in which the first lead portion 42bL extends from the first electrode portion 42bE, and averaging the measured values of the width. The maximum width W1 of the first electrode portion 42bE is measured in a direction parallel to the width direction (the direction of W2).

For example, as shown in FIGS. 5 and 6, one end of the first lead portion 42bL is connected to the first electrode portion 42bE in such a manner that an overlapping region 42bt is provided at an end portion of the first electrode portion 42bE. This overlapping region 42bt is considered as a portion of the first electrode portion 42bE.

As shown in FIGS. 5 and 6, the first solid electrolyte body 42dx has an uneven portion 42ds at least on a surface region facing the first electrode portion 42bE (in FIGS. 5 and 6, the upper surface of the first solid electrolyte body 42dx). The uneven portion 42ds has a plurality of protrusions 42dr. The first electrode portion 42bE is in direct contact with the uneven portion 42ds.

Also, an insulating layer 43 having an uneven surface 43s is formed on the surface of the first solid electrolyte body 42dx facing the first lead portion 42bL (in FIGS. 5 and 6, a portion of the upper surface of the first solid electrolyte body 42dx located on a left rear end side and a rear-end-side side surface which is continuous with that portion of the upper surface). The first lead portion 42bL is in direct contact with the insulating layer 43.

Notably, “on the surface” of the solid electrolyte body in the claims means that the insulating layer 43, which is separate from the first solid electrolyte body 42dx, is further formed on the “surface of the first solid electrolyte body 42dx” so as to cover the surface of the first solid electrolyte body 42dx.

Notably, in the present embodiment, the uneven portion 42ds is formed on the entire upper surface of the first solid electrolyte body 42dx facing the first electrode portion 42bE, and on all the four side surfaces of the first solid electrolyte body 42dx. However, the uneven portion 42ds may be formed only in a region of the upper surface of the first solid electrolyte body 42dx, which region faces the first electrode portion 42bE (namely, a region overlapping with the first electrode portion 42bE). Also, the uneven portion may be formed on the lower surface of the first solid electrolyte body 42dx facing the second electrode portion 42aE.

In the present embodiment, the insulating layer 43 is formed in a region whose width is greater than the width of the first lead portion 42bL, and the insulating layer 43 projects from the opposite edges of the first lead portion 42bL in the width direction. By virtue of this configuration, even in the case where printing misalignment or the like occurs when a green lead which is to become the first lead portion 42bL is formed on the surface of the insulating layer 43 by means of printing or the like during manufacture, the first lead portion 42bL can be formed on the surface of the insulating layer 43 without fail.

FIG. 8 shows the effects achieved by the uneven portion 42ds and the surface 43s.

Since the first electrode portion 42bE containing Au is high in surface tension, and the degree of adhesion between the first electrode portion 42bE and the first solid electrolyte body 42dx is low. In order to overcome such a drawback, the uneven portion 42ds is provided on the first solid electrolyte body 42dx. As a result, the first electrode portion 42bE tightly adheres to the uneven portion 42ds due to the anchor effect, whereby the adhesion between the first electrode portion 42bE and the first solid electrolyte body 42dx is enhanced.

The first lead portion 42bL containing Au is also high in surface tension, and the degree of adhesion between the first lead portion 42bL and its counterpart member is low. In order to overcome such a drawback, the uneven surface 43s is provided on the insulating layer 43. As a result, the first lead portion 42bL tightly adheres to the surface 43s due to the anchor effect, whereby the adhesion between the first lead portion 42bL and the insulating layer 43, which is the counterpart member, is enhanced.

Further, since the insulating layer 43 is present between the first lead portion 42bL and the first solid electrolyte body 42dx, the first lead portion 42bL does not come into contact with the first solid electrolyte body 42dx in the first ammonia sensor section 42x, which is a mixed-potential-type sensor (detection cell).

Therefore, it is possible to prevent occurrence of an electrode reaction ER (FIG. 8), via the first solid electrolyte body 42dx, between the first lead portion 42bL and the reference electrode portion 42aE facing the first lead portion 42bL, thereby preventing deterioration of detection accuracy.

The uneven portion 42ds can be formed as follows, for example. First, a material obtained by mixing together a binder and a calcinated powdery material for the first solid electrolyte body 42dx is pressure-formed into a rectangular-plate-shaped base. Subsequently, a material obtained by mixing together a binder and spherical particles prepared by granulating a powdery material for the first solid electrolyte body 42dx and having a predetermined particle size is applied to the upper surface and four side surfaces of the base. When the entire base is fired after that, the spherical particles are appropriately joined to one another as a result of sintering. However, some spherical particles are exposed to the surface of the first solid electrolyte body 42dx, so that the surface becomes uneven.

Like the uneven portion 42ds, the uneven surface 43s may be formed to have an uneven shape by preparing a material by mixing together a binder and spherical particles, which are prepared by granulating a powdery material for the insulating layer 43, and applying the prepared material to an even (smooth) surface of the first solid electrolyte body 42dx, followed by firing.

However, in the case where the insulating layer 43 is formed on the even (smooth) surface of the first solid electrolyte body 42dx, the degree of adhesion between the first solid electrolyte body 42dx and the insulating layer 43 may decrease, or a process of manufacturing the insulating layer 43 may become complex.

In view of the above, as shown in FIG. 8, the insulating layer 43 is formed on the surface of the uneven portion 42ds in such a manner that the surface 43s has an uneven shape which follows the contour of the uneven portion 42ds. This is preferable, because the adhesion between the first solid electrolyte body 42dx and the insulating layer 43 is also enhanced.

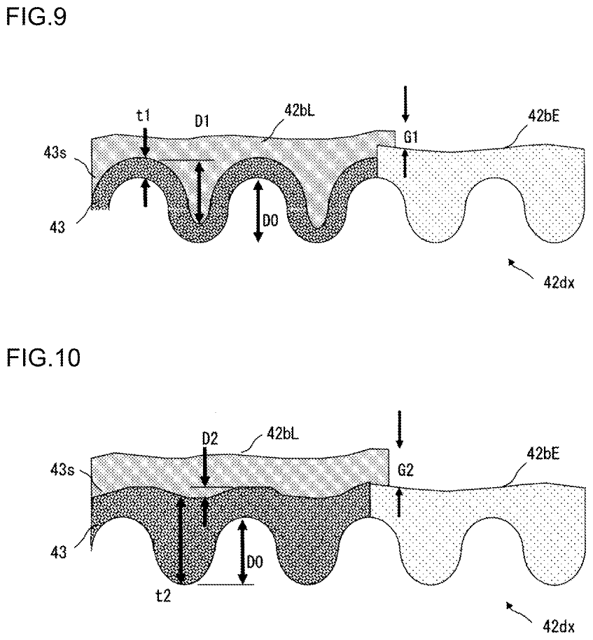

Herein, the expression “follows” means that the surface 43s has a shape which resembles (reflects) the shape of the uneven portion 42ds and which is approximately the same as that of the uneven portion 42ds. Namely, as shown in FIG. 9, the thickness t1 of the insulating layer 43 is not large enough to fill the difference in height D0 of the uneven portion 42ds, and the difference in height D1 of the uneven surface 43s is close to the difference in height D0 of the uneven portion 42ds.

Also, since the thickness t1 of the insulating layer 43 is not larger than the difference in height D0, a step between the uneven portion 42ds, which serves as a groundwork for the first electrode portion 42bE, and the surface 43s, which serves as a groundwork for the first lead portion 42bL, becomes small. As a result, a step G1 of a connection portion between the first electrode portion 42bE and the first lead portion 42bL also becomes small, and therefore, occurrence of wire breakage between the first electrode portion 42bE and the first lead portion 42bL can be prevented.

In contrast, as shown in FIG. 10, in the case where the thickness t2 of the insulating layer 43 is large enough to fill the difference in height D0 of the uneven portion 42ds, the difference in height D2 of the uneven surface 43s becomes smaller than the difference in height D0 of the uneven portion 42ds, and the uneven shape of the surface 43s becomes different from the uneven shape of the uneven portion 42ds; i.e., the surface 43s becomes flat.

As a result, the anchor effect for tightly adhering the first lead portion 42bL to the surface 43s becomes weak, and it becomes difficult to enhance the adhesion between the first lead portion 42bL and the insulating layer 43, which is a counterpart member.

Also, since the thickness t2 of the insulating layer 43 is larger than the difference in height D0, the step between the uneven portion 42ds, which serves as a groundwork for the first electrode portion 42bE, and the surface 43s, which serves as a groundwork for the first lead portion 42bL, becomes large. As a result, the step G2 of the connection portion between the first electrode portion 42bE and the first lead portion 42bL becomes large, and therefore, wire breakage may occur between the first electrode portion 42bE and the first lead portion 42bL.

In particular, in order to obtain a “following” shape, it is preferred that the thickness t1 of the insulating layer 43 on the surface of the uneven portion 42ds be smaller than the difference in height D0 of the uneven portion 42ds as shown in FIG. 9, because the surface 43s has an uneven shape which reflects the shape of the uneven portion 42ds without fail.

The thickness t1 of the insulating layer 43 is determined by obtaining, as a section as shown in FIG. 8, a 600x sectional SEM image along the direction in which the first lead portion 42bL extends, and measuring the maximum thickness of the insulating layer 43 in that image as the thickness t1.

Also, the difference in height D0 of the uneven portion 42ds is determined as follows. As a section as shown in FIG. 8, a 600x sectional SEM (Scanning Electron Microscope) image along the direction in which the first lead portion 42bL extends is obtained at three different positions in the width direction of the first lead portion 42bL. The maximum value of the difference in height between a convex portion (protrusion 42dr) of the uneven portion 42ds and a concave portion thereof located adjacent to the convex portion in each image is used as the difference in height D0.

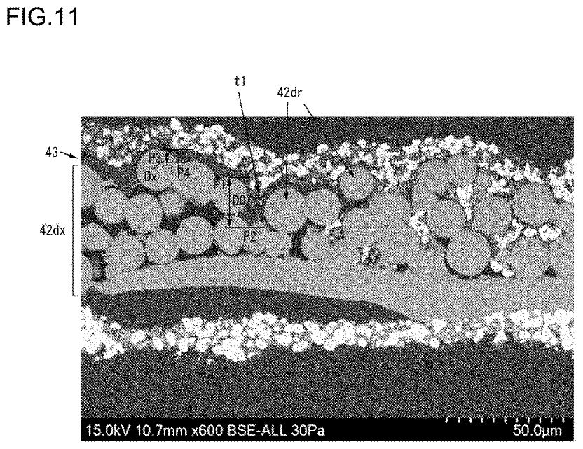

FIG. 11 shows an actual sectional SEM image along the direction in which the first lead portion 42bL extends. In the example of FIG. 11, the protrusions 42dr constituting the uneven portion 42ds are spherical.

The difference in height D0 of the uneven portion 42ds is the maximum value of the difference in height between a convex portion and a concave portion located adjacent to each other. In FIG. 11, the difference in height Dx is small at portions P3 and P4 where adjacent spheres are joined together as a result of sintering and the individual spherical shapes are lost.

Meanwhile, at portions P1 and P2 where adjacent spheres are not joined together, a gap is present therebetween, and the individual spherical shapes are maintained, the difference in height D0 is large. Therefore, the difference in height D0 at the portions P1 and P2 is considered to represent the difference in height of the uneven portion 42ds.

Namely, the portions P1 and P2—where adjacent spheres are not joined together, a gap is present therebetween, and the individual spherical shapes are maintained—reflect the uneven shape of the uneven portion 42ds.

Notably, in FIG. 11, the thickness t1 of a region of the insulating layer 43 corresponding to the portions P1 and P2 is the maximum thickness of the insulating layer 43, and the thickness t1 of that region is considered to represent the thickness of the insulating layer 43.

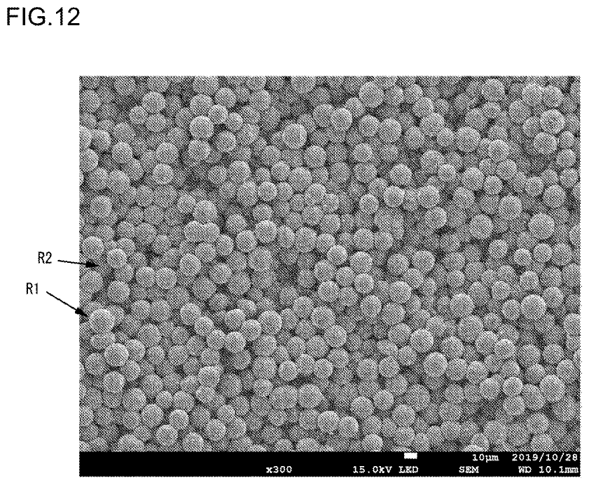

FIG. 12 shows an actual SEM image of the surface of the uneven portion 42ds. It can be seen from the SEM image that the protrusions 42dr constituting the uneven portion 42ds are a plurality of spherical protrusions.

When the mean particle diameter of the spherical protrusions is 10 to 50 μm, the anchor effect can be obtained without fail, and the difference in height of the uneven portion is not excessively large. Therefore, the step G1 of the connection portion between the first electrode portion 42bE and the first lead portion 42bL becomes small, and occurrence of wire breakage between the first electrode portion 42bE and the first lead portion 42bL can be prevented further reliably.

When the mean particle diameter of the spherical protrusions is less than 10 μm, the anchor effect may become insufficient in some cases. When the mean particle diameter of the spherical protrusions exceeds 50 μm, the difference in height of the uneven portion becomes excessively large. Therefore, the step G1 of the connection portion between the first electrode portion 42bE and the first lead portion 42bL becomes large, and wire breakage may occur between the first electrode portion 42bE and the first lead portion 42bL.

Notably, the mean particle diameter of the spherical protrusions is determined as follows. 10 particles appearing on the outermost surface in a 300x sectional SEM image shown in FIG. 12 are extracted, and the average of equivalent circle diameters of the extracted particles is obtained as the mean particle diameter. At that time, for a particle which is partially hidden behind surrounding particles, its diameter may be obtained by considering the contour of an unhidden portion of the particle as an arc.

Also, in the case where the uneven portion 42ds is also formed in a region of the surface of the first solid electrolyte body 42dx other than the regions corresponding to the first electrode portion 42bE and the first lead portion 42bL, the measurement is performed by obtaining a surface SEM image as shown in FIG. 12 from that region.

Meanwhile, in the case where the uneven portion 42ds is formed on only a region of the surface of the first solid electrolyte body 42dx, which region is covered by the first electrode portion 42bE, the first electrode portion 42bE is dissolved using aqua regia, and the measurement is performed by obtaining a surface SEM image as shown in FIG. 12 from the region from which the first electrode portion 42bE has been removed.

The multi-gas sensor element (the multi-gas sensor element section 100A) of the present invention includes at least one sensor element (the ammonia sensor section 42x) and one NOx sensor section.

In the case where the multi-gas sensor element includes two or more sensor elements (the ammonia sensor sections 42x and 42y), each of all the sensor elements includes the uneven portion 42ds and the insulating layer 43 described above.

Needless to say, the present invention is not limited to the above-described embodiment and encompasses various modifications and equivalents within the idea and scope of the present invention.

The sensor element is not limited to that provided in a multi-gas sensor element and may be configured as a sole ammonia sensor element.

Also, the position at which the sensor element is provided in the multi-gas sensor element is not limited to the above-described position, and, for example, the ammonia sensor sections 42x and 42y may be provided on the surface of the insulating layer 23e which forms the outer surface (upper surface) of the NOx sensor section 30A.

No limitation is imposed on the manner of disposing the first electrode and the second electrode, so long as the first electrode and the second electrode are disposed to face each other at least partially while sandwiching the solid electrolyte body.

The surface shapes of the uneven portion and the insulating layer are not limited to the above-described shapes.

### DESCRIPTION OF REFERENCE NUMERALS

30A: NOx sensor section
42ax, 42ay: second electrode (first reference electrode and second reference electrode)
42bx, 42by: first electrode (first detection electrode and second detection electrode)
42bE: first electrode portion
42bL: first lead portion
42dx: solid electrolyte body (first solid electrolyte body)
42dr: protrusion
42ds: uneven portion
42x,42y: sensor element (first ammonia sensor section and second ammonia sensor section)
43: insulating layer
43s: uneven surface
100A: multi-gas sensor element (multi-gas sensor element section)
138: metallic shell
200A: gas sensor (multi-gas sensor)
t1: thickness of the insulating layer
D0: difference in height of the uneven portion

1. A sensor element comprising:
a first electrode containing Au as a main component;
a second electrode; and
a solid electrolyte body including an insulating layer with a surface that has an uneven shape, wherein
the first electrode and the second electrode are disposed to face each other at least partially while sandwiching the solid electrolyte body therebetween,
the first electrode has a first electrode portion and a first lead portion,
the first electrode portion faces the second electrode while sandwiching the solid electrolyte body in cooperation with the second electrode,
the first lead portion is electrically connected to the first electrode portion and extends from the first electrode portion,
the solid electrolyte body has an uneven portion in a surface region facing the first electrode portion, the uneven portion having a plurality of protrusions;
the first electrode portion is in direct contact with the uneven portion,
the insulating layer is formed on a surface of the solid electrolyte body facing the first lead portion, and
the first lead portion is in direct contact with the insulating layer.

2. The sensor element according to claim 1, wherein the uneven shape of the insulating layer corresponds to a contour of the uneven portion.

3. The sensor element according to claim 1, wherein
the uneven portion is also formed in a surface region of the solid electrolyte body facing the first lead portion, and
the insulating layer has a thickness smaller than a difference in height of the protrusions.

4. The sensor element according to claim 1, wherein the first electrode contains a component of the solid electrolyte body.

5. The sensor element according to claim 1, wherein the protrusions are spherical protrusions, each of which has a mean particle diameter of 10 to 50 μm.

6. The sensor element according to claim 1, wherein
the second electrode contains Pt as a main component,
the first electrode serves as a detection electrode,
the second electrode serves as a reference electrode, and
the first electrode, the second electrode, and the solid electrolyte body constitute a mixed-potential-type ammonia detection cell.

7. A multi-gas sensor element comprising:
the sensor element according to claim 6; and
an NOx sensor section for measuring the concentration of nitrogen oxides in a gas under measurement.

8. A gas sensor comprising:
the sensor element according to claim 1; and
a metallic shell which holds the sensor element.
